(19) United States
(12) Patent Application Publication
(10) Pub. No.: US 2026/0355066 A1
(43) Pub. Date: Nov. 12, 2026

(54) SYSTEM AND METHODS FOR NEONATAL GASTRIC TUBE FEEDING AND VENTING

(71) Applicant: New York University, NEW YORK, NY (US)

(72) Inventors: Logan Groneck HART, New York, NY (US); Sean BAILEY, Roslyn, NY (US); Mladen BARBIC, Sterling, VA (US); Vadim GORDIN, Brooklyn, NY (US)

(21) Appl. No.: 19/125,113

(22) PCT Filed: Nov. 6, 2023
(86) PCT No.: PCT/US2023/036884

§ 371 Date: Apr. 28, 2025

Related U.S. Application Data
(60) Provisional application No. 63/382,695, filed on Nov. 7, 2022.
(60) Provisional application No. 63/484,853, filed on Feb. 14, 2023.

(51) Int. Cl. A61J 15/00

(52) U.S. Cl. CPC A61J 15/0003; A61J 15/0073; A61J 15/0076; A61J 15/0084; A61J 15/0088

(57) ABSTRACT

A device, system and method are for providing a feeding and aspiration to a patient. The device includes a catheter extending longitudinally from a proximal end to a distal end; a feed lumen extending longitudinally through the catheter and including a feed opening extending through an exterior wall of the catheter along a distal portion thereof so that the feed lumen is open to an exterior of the catheter via the feed opening; an aspiration lumen extending longitudinally through the catheter and separated from the feed lumen along a length thereof via a separating wall, the aspiration lumen including aspiration openings extending laterally through the exterior wall along the distal portion, so that the aspiration lumen is open to the exterior via the aspiration openings; and a sensor positioned in communication with the aspiration lumen and configured to detect a change in state within the aspiration lumen.

### PRIORITY CLAIM

The present disclosure claims priority to U.S. Provisional Patent Application Ser. No. 63/382,695 filed Nov. 7, 2022 and 63/484,853 filed Feb. 14, 2023; the disclosures of both applications are incorporated herewith by reference.

### BACKGROUND

Most patients admitted into the Neonatal Intensive Care Unit (NICU) are newborns that are preterm, have low birth weight (less than 2.5 kg), or have a health condition requiring specialized care. Many of these patients require respiratory and/or nutritional support which may be provided via nasally administered non-invasive ventilation and an orogastric feeding tube, respectively. Neonates with underdeveloped feeding coordination, or with lower esophageal tone from an immature nervous system, however, may ingest large volumes of pressurized air through the nose, resulting in gaseous distension that increases the risk of feeding intolerance. The resulting physical exam findings may promote provider uncertainty regarding gastrointestinal health.

In some cases, this necessitates the interruption of feeding within just a few days of providing non-invasive respiratory support, requiring additional hospitalization and additional days of intravenous (IV) nutrition, all of which can increase the risk of infection. Where gaseous abdominal distension impairs lung volume, an infant is intolerant of the non-invasive ventilation, the patient may have to be intubated, increasing the risk of chronic lung disease. The resulting abdominal distension and inability to tolerate feed may also result in diagnostic uncertainty that requires abdominal radiographs which expose the patient to additional radiation. Increased gastrointestinal pressures have been hypothesized to increase translocation of bacteria and impair mesenteric blood flow as possible mechanisms for the development of feeding intolerance and necrotizing enterocolitis.

Currently, NICU nurses may attempt to treat gaseous distension by pulling air through the orogastric tube between feeds. However, in many cases, nurses are unable to vent enough air as there is often a very limited break (e.g., 30 minutes) between feedings, if at all. In addition, pulling air through the orogastric tube is not an option for cases in which the tip is submerged in the feed. Decreasing ventilator pressures to reduce gastric insufflation, prone positioning and rectal stimulation are all attempted to relieve the burden of gaseous abdominal distension with limited efficacy.

### SUMMARY

The present disclosure relates to a device for providing a feeding and aspiration to a patient. The device includes a catheter extending longitudinally from a proximal end to a distal end; a feed lumen extending longitudinally through the catheter and including at least one feed opening extending through an exterior wall of the catheter along a distal portion thereof so that the feed lumen is open to an exterior of the catheter via the at least one feed opening; an aspiration lumen extending longitudinally through the catheter and separated from the feed lumen along a length thereof via a separating wall, the aspiration lumen including a plurality of aspiration openings extending laterally through the exterior wall of the catheter along the distal portion, so that the aspiration lumen is open to the exterior of the catheter via the aspiration openings; and a sensor positioned in communication with the aspiration lumen and configured to detect a change in state therewithin.

In an embodiment, the sensor includes a plurality of electrodes configured to measure an impedance within the aspiration lumen.

In an embodiment, each of the plurality of electrodes is configured as a wire electrode embedded in one of the separating wall and the exterior wall so that, a distal portion of which is exposed to the interior of the aspiration lumen, immediately proximal of the distal portion of the catheter.

In an embodiment, the device further includes a third lumen extending through the catheter, the plurality of electrodes extending through the third lumen so that distal portions thereof extend through a wall separating the third lumen from the aspiration lumen so that distal portions of the electrodes are exposed to an interior of the aspiration lumen.

In an embodiment, the aspiration lumen extends from the proximal end of the catheter to a distal end that is proximal a distal end of the feed lumen.

In an embodiment, a cross-section of the feed lumen is substantially circular.

In an embodiment, the aspiration lumen extends about a portion of a periphery of the feed lumen.

In addition, the present disclosure relates to a system for providing a feeding and aspiration to a patient. The system includes a catheter extending longitudinally from a proximal end to a distal end, the catheter including a feed lumen and an aspiration lumen, the feed lumen extending longitudinally through the catheter and including at least one feed opening extending through an exterior wall of the catheter along a distal portion thereof so that the feed lumen is open to an exterior of the catheter via the at least one feed opening, the aspiration lumen extending longitudinally through the catheter and separated from the feed lumen along a length thereof via a separating wall, the aspiration lumen including a plurality of aspiration openings extending laterally through the exterior wall of the catheter along the distal portion so that the aspiration lumen is open to the exterior of the catheter via the aspiration openings; a sensing mechanism configured to detect a change in state within the aspiration lumen; and a controller configured to control a pressure of the aspiration lumen based on a detected change in state within the aspiration lumen.

In an embodiment, the sensing mechanism includes a plurality of electrodes positioned along the aspiration lumen to detect the change in state therein, and an impedance sensor configured to receive input from the plurality of electrodes and to output impedance data indicative of an internal state of the aspiration lumen.

In an embodiment, the system further includes a first pump configured to provide a flow of liquid feed distally through the feed lumen and out of the at least one feed opening.

In an embodiment, the system further includes a second pump configured to control the pressure of the aspiration lumen.

In an embodiment, the first pump is a syringe pump.

In an embodiment, the second pump is configured to provide a negative pressure through the aspiration lumen during an aspiration and a positive pressure therethrough when the impedance data indicates a change in state within the aspiration lumen from a gas to a liquid.

In an embodiment, the second pump is a peristaltic pump.

In an embodiment, the system further includes a pressure sensor positioned at a proximal end of the catheter for measuring a gastric pressure.

Furthermore, the present disclosure relates to a method for providing independent and simultaneous feeding and aspiration to a premature neonate. The method includes inserting a catheter into a stomach of the premature neonate; providing a liquid feed to the premature neonate via a feed lumen extending longitudinally through the catheter, the liquid feed passing distally through the feed lumen and laterally out of the feed lumen via at least one feed opening extending through an exterior wall of the catheter; activating, via a controller, an aspiration through an aspiration lumen extending longitudinally through the catheter and via a plurality of aspiration openings extending through an exterior wall of the catheter to draw excess gas through the plurality of aspiration openings, proximally out of the aspiration lumen; continuously determining, via the controller, an impedance data indicating an internal state of the aspiration lumen; and when the impedance data indicates a change in state within the aspiration lumen from a gas to a liquid, reversing a flow of pressure through the aspiration lumen to clear the aspiration lumen.

In an embodiment, the aspiration is controlled based on whether a measured gastric pressure exceeds a predetermined threshold value.

In an embodiment, the impedance data is determined based on input received from a plurality of electrodes, a portion of which is positioned along an interior of the aspiration lumen.

In an embodiment, a reversal of flow to clear the aspiration lumen ends when the impedance data indicates that liquid is no longer detected in the aspiration lumen.

In an embodiment, the method further includes reactivating a negative pressure through the aspiration lumen after a pause.

In an embodiment, the pause is determined via one of a predetermined delay window and a measured gastric pressure.

In an embodiment, the method further comprises overriding a reversal of flow in the aspiration lumen for variable periods of time, based on one of user input and a gastric pressure threshold.

In an embodiment, the gastric pressure threshold value is automatically calibrated based on historical impedance measurements.

### BRIEF DESCRIPTION

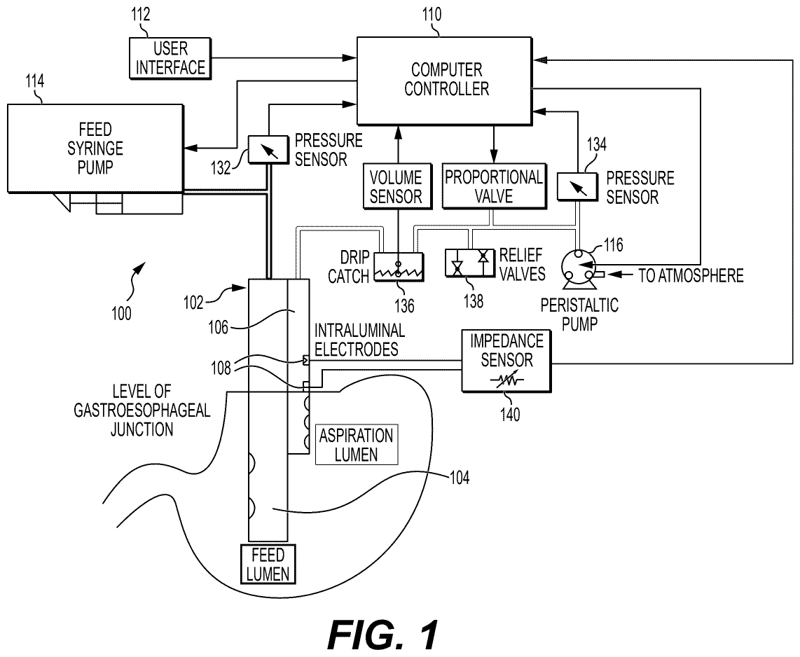

FIG. 1 shows a schematic diagram of a system according to an exemplary embodiment of the present disclosure;

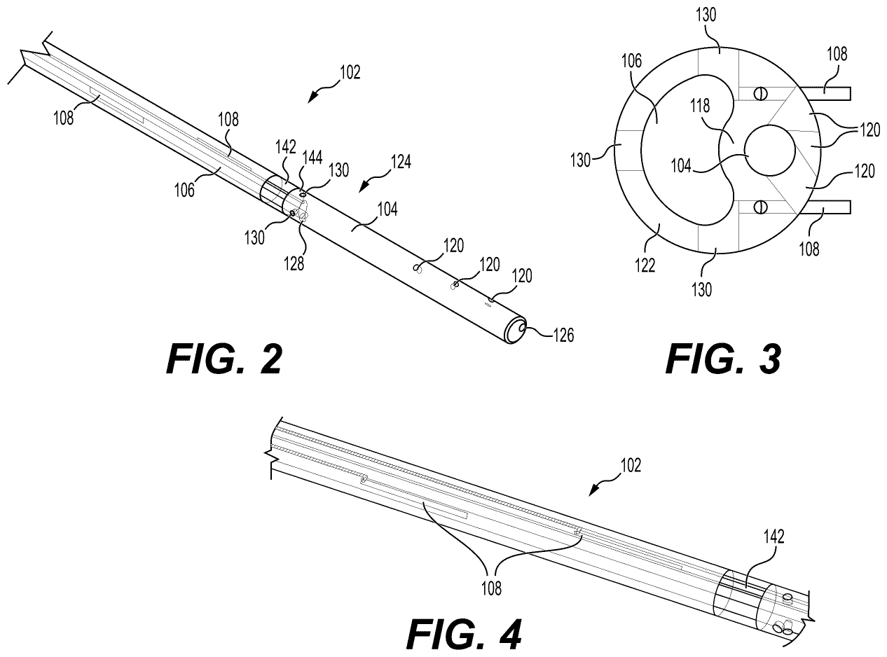

FIG. 2 shows a perspective view of a distal portion of a catheter according to the exemplary system of FIG. 1;

FIG. 3 shows a cross-sectional view of the distal portion of the catheter of FIG. 2;

FIG. 4 shows a perspective view of a portion of the catheter including electrodes for sensing an internal state of an aspiration lumen of the catheter according to the system of FIG. 1;

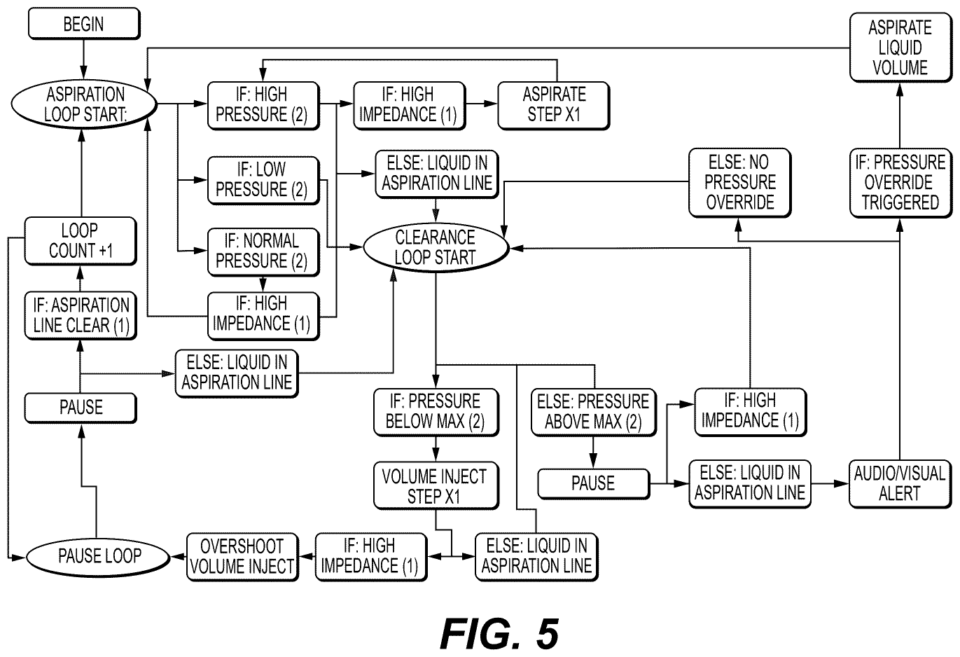

FIG. 5 shows a flow chart of an exemplary method according to the system of FIG. 1;

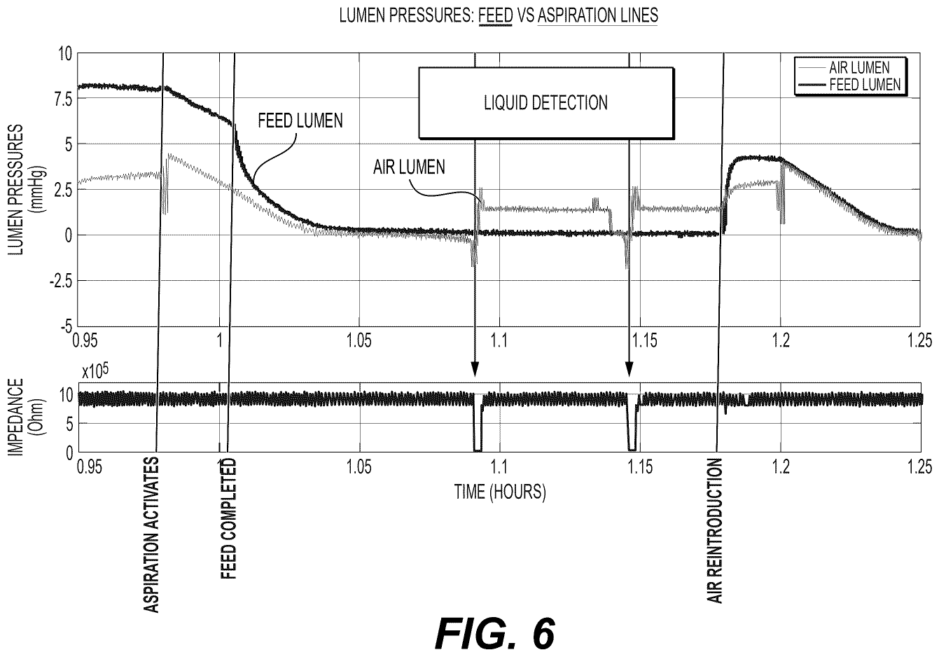

FIG. 6 illustrates a graph showing an 18-minute data sample of gastric pressures and corresponding impedance data collected during a study of the system according to FIG. 1, under physiologic run conditions; and

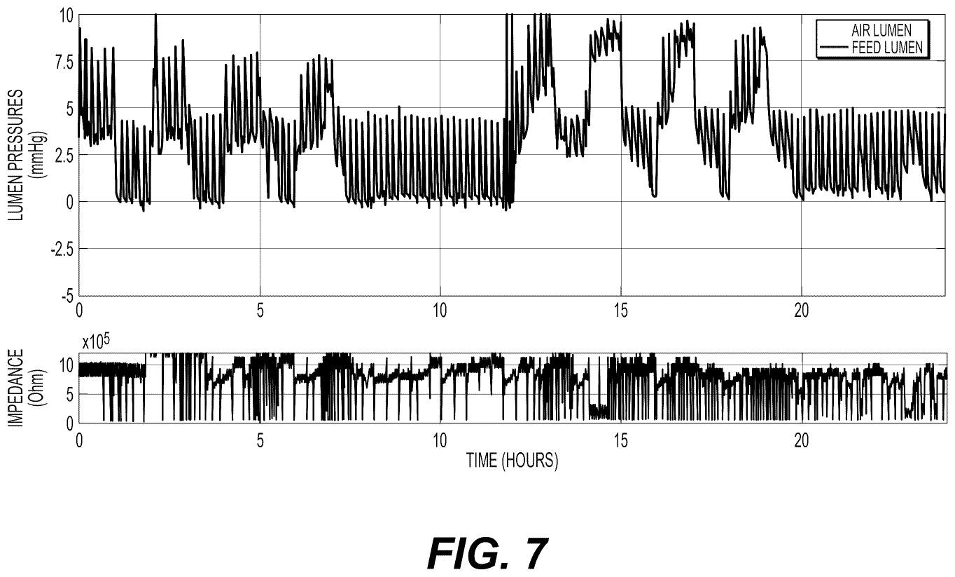

FIG. 7 illustrates a graph showing a 24-hour data sample of gastric pressure and corresponding impedance data collected during the study of the system according to FIG. 1; and

### DETAILED DESCRIPTION

The present disclosure may be further understood with reference to the following description and the appended drawings, wherein like elements are referred to with the same reference numerals. The present disclosure relates to gastric feeding systems and methods and, in particular, relates to a gastric feeding system and method configured to provide independent and simultaneous feeding and air evacuation for premature neonates. Exemplary embodiments of the present disclosure describe a system and method comprising a dual lumen feeding catheter configured to be inserted into a stomach of a patient in a manner substantially similar to traditional orogastric feeding tubes.

The dual lumen catheter of the present disclosure, however, includes a first lumen configured to provide a liquid feed to a patient and a second lumen configured to provide an aspiration to remove excess gas from the stomach. The aspiration lumen is in communication with a sensor to determine the state of an interior of the aspiration lumen—e.g., to detect when a liquid is within the aspiration lumen. Detection of a liquid in the aspiration lumen indicates that excess gas has been evacuated and only gastric contents remain. Thus, when liquid is detected in the aspiration lumen, a flow through the aspiration lumen is reversed, providing a positive pressure therein to clear any blockages therein. It should be noted that the terms “proximal” and “distal,” as used herein are intended to refer to a direction toward (proximal) and away from (distal) a user of the system.

As shown in FIGS. 1-4, a system 100 for providing feed to a premature neonate comprises a dual lumen catheter 102 configured to be inserted through a patient mouth and into a stomach. The catheter 102 includes a feed lumen 104 for providing a liquid feed to a patient and an aspiration lumen 106 for providing an aspiration to remove excess gas. A negative pressure gradient may be independently and simultaneously provided through the aspiration lumen 106 to remove excess gas within the stomach to prevent gaseous distension while feed is being provided to the patient via the feed lumen 104. The aspiration lumen 106 is in communication with a sensor configured to sense a change in state within the aspiration lumen 106.

In an exemplary embodiment, the sensor includes electrodes 108 configured to continuously monitor an impedance data therewithin. Changes in the impedance data indicate when there is a change in state within the aspiration lumen 106—e.g., a change from gas to liquid. Detection of a liquid within the aspiration lumen 106 indicates that all excess gas has been evacuated and only gastric contents remain. Thus, flow through the aspiration lumen 106 is briefly reversed to provide a positive pressure gradient therein, clearing any blockages. The system 100 further comprises a processor or computer controller 110 configured to receive and analyze the data measured via the electrodes 108 and to control a pump device, which includes a first pump 114 for providing flow through the feed lumen 104 and a second pump 116 for providing flow through the aspiration lumen 106.

The catheter 102, as shown in FIG. 2-4, extends longitudinally from a proximal end to a distal end and includes the feed lumen 104 and the aspiration lumen 106 extending longitudinally therethrough. The catheter 102 may have the same dimensions as traditional orogastric feeding tubes and is sized, shaped, and configured to be passed through the mouth, down an oropharynx and esophagus of a patient and into a stomach. According to an exemplary embodiment, the catheter 102 may be a 6 Fr catheter and have a distal portion 124 configured to be received within the stomach of the patient. It will be understood by those of skill in the art, however, that the catheter 102 may vary in size and may have size ranging between, for example, 4 French and 8 French.

As shown in FIG. 3, the feed lumen 104 and the aspiration lumen 106 extend through the catheter 102 such that they are separated via a wall 118 so that they are not in fluid communication with one another. In an exemplary embodiment, a cross-sectional area of the feed lumen 104 is substantially circular while the aspiration lumen 106 extends along a length of the catheter 102, about a portion of a periphery of the feed lumen 104 so that a cross-section thereof has a substantially crescent moon-shape. It will be understood by those of skill in the art, however, that the feed and aspiration lumens 104, 106 may have any of a variety of shapes so long as the feed and aspiration lumens 104, 106 are configured to provide feed and aspiration to the patient, as will be described in further detail below.

The feed lumen 104 extends through the catheter 102, substantially along an entire length thereof, so that a distal end 126 thereof corresponds to a distal end of the catheter 102. The feed lumen 104 is open to an exterior of the catheter 102 via a plurality of openings 120 extending laterally through an exterior wall 122 of the catheter 102, along a distal portion 124 of the catheter 102. Liquid feed may be passed distally through the feed lumen 104 out of the plurality of openings 120 to be provided to the patient. The proximal end of the catheter 102 which, in the operative configuration, remains external to the patient, may include a Y-connector for directing the appropriate flow to the feed lumen 104 and/or aspiration lumen 106. According to an exemplary embodiment, the Y-connector may include a pressure sensor 132 in-line with the feed lumen 104 for measuring and recording gastric pressures during and immediately after the process of feed administration through a column of liquid feed. A user (e.g., nurse or physician) may input a feeding rate at which feed may be provided to the patient. Feed may be provided via, for example, a syringe pump, either as a single device in combination with an air aspirating pump or as a standalone device, as will be understood by those of skill in the art.

The aspiration lumen 106 extends through a length of the catheter 102 to a distal end 128 that may be proximal to a distal end 126 of the feed lumen 104. Similarly to the feed lumen 104, the aspiration lumen 106 includes a plurality of openings 130 extending through the exterior wall 122 of the catheter 102 along the distal portion 124, via which the aspiration lumen 106 is open to an exterior of the catheter 102 and via which aspiration may be provided. Some of the openings 130 may be positioned along the aspiration lumen 106 such that, when the distal portion 124 of the catheter 102 is in an operative position within the stomach, the openings 130 are positioned within proximity to and extending distally from the gastroesophageal junction of the patient. In an exemplary embodiment, the openings 130 may be longitudinally aligned relative to one another. In another exemplary embodiment, the openings 130 may be positioned circumferentially about the catheter 102, at a single point along a length thereof, to limit issues of capillary action which may contribute to liquid aspiration and poor clearance of distal ones of the openings 130. It will be understood by those of skill in the art, however, that the openings 130 may be distributed about and/or along the catheter 102 in any of a variety of configurations so long as the openings 130 are positioned to permit excess gas within the patient stomach to be suctioned therethrough, and proximally through the aspiration lumen 106 to be removed from the body. It will also be understood by those of skill in the art that suctioning of liquid gastric contents would result in detection of a state change via the electrodes 108, as will be described in further detail below.

According to an exemplary embodiment, the degree and/or amounts of time during which the excess gas may be removed from the stomach may be adjusted based on the particular patient's needs. For example, a preterm infant with lower pressure non-invasive ventilatory support requirements may require just a small amount of continuous gas removal while an infant with higher pressure non-invasive ventilatory support requirements may require intermittent gas removal. In an exemplary embodiment, gastric pressure data may be used to determine the degree/amount of gas removal and/or limit the degree/amount of gas removal to maintain desired pressures within the gastric system. In another embodiment, users may select preset modes such as, for example, intermittent suctioning, or may select a frequency or different categories such that may be present. Preset categories may be indicated with, for example, labels indicating intensity (e.g., low, medium, high) or with colored coding. As will be described in further detail below, these selections may be adjusted and/or input via, for example, a user interface 112.

As described above, the aspiration lumen 106 may be in communication with one or more sensors for detecting a change in state within the aspiration lumen 106. In an exemplary embodiment, the one or more sensors may include, for example, electrodes 108 in communication with and/or positioned along the aspiration lumen 106 for determining an impedance within the aspiration lumen 106 using an impedance sensor 140, and communicating such impedance data to the controller 110. The electrodes 108 may be positioned immediately proximal of the distal portion 124 including the openings 130. In an exemplary embodiment, as shown in FIG. 4, the electrodes 108 may be embedded within the walls of the catheter 102—e.g., the exterior wall 122 and/or the wall 118 separating the feed lumen 104 from the aspiration lumen 106—such that a portion thereof is exposed to an interior of the aspiration lumen 106. In an exemplary embodiment, the electrodes 108 are configured as wire electrodes, distal portions of which are exposed to the interior of the aspiration lumen 106, as shown in FIG. 3. Although the exemplary embodiments show and describe the electrodes 108 as extending through a wall of the catheter 102, in another embodiment, the catheter 102 may include a third lumen through which the electrodes 108 may extend to, for example, the controller 110. In this embodiment, the distal portions of the electrodes 108 would also be exposed to the interior of the aspiration lumen 106 to enable monitoring of the impedance therein.

The electrodes 108 enable continuous monitoring of impedance data that is communicated to the controller 110, which may be external to the patient at, for example, bedside. This impedance data is used to indicate the internal state of the aspiration lumen 106 during operation. Detected changes in magnitude and/or phase of impedance drops (e.g., beyond a threshold value) for certain durations indicate to the controller 110 when a change from air to liquid interface has occurred, such as is expected when excess air has been evacuated leaving only gastric contents remaining. It will be understood by those of skill in the art that the impedance data includes an electrical impedance defined via the ratio of voltage across a liquid (e.g., feed) and current through the same liquid. This electrical impedance has a magnitude and phase value, both of which may vary with frequency, and all of which may be measured. In an exemplary embodiment, the impedance may be measured from DC to any higher frequency such as, for example, 0 to 250 kHZ. Impedance measurements may occur continuously or intermittently.

Following successful removal of air to achieve gastric decompression, an impedance drop would signal to the controller 110 to instruct the pump device to briefly reverse flow of the second pump 116, which drives flow through the aspiration lumen 106. In other words, negative pressure applied through the aspiration lumen 106 to remove excess gas is reversed in direction to create a temporary positive pressure to clear the aspiration lumen 106, preventing blockage and minimizing loss of gastric contents, until such a time when the system 100 resumes application of negative pressure to attempt aspiration of any interval accumulation of gastric air. Modification of intralumenal electrode 108 spacing and coded parameters for detected impedance drops may be utilized to modify the volume of liquid aspirate required to activate pressure reversal and line clearance. In an exemplary embodiment, the aspiration lumen 106 includes two electrodes 108 for measuring impedance data. It will be understood by those of skill in the art, however, that the aspiration lumen 106 may include any number of electrodes 108 so long as the electrodes are configured to measure impedance and/or detect the contents of the aspiration lumen 106 as described below.

Intralumenal impedance sensors allow for monitoring of gastric aspirates for gaseous or liquid interfaces within the tubing to direct the automation of an external pump system. This minimizes loss of gastric contents that may lead to nutritional and electrolyte deficiencies seen in other continuous or intermittent pumping gastric decompression systems. It further limits the frequency of tubing obstruction through continuous monitoring to ensure that the line remains clear, a feature not possible with external catheter impedance monitors. Other technologies largely focus on monitoring of the gastric environment to ensure proper tube placement or approximate stomach volumes, where the system 100 provides information related to successful functioning of the catheter 102. Other dual lumen catheters for decompression, for example, rely on administering feeds distal to the stomach (such as in the duodenum) to prevent loss of feeding during gastric aspiration and decompression. The system 100 maintains more physiologic nutrition delivery through simultaneous feeding and decompression.

Although the exemplary embodiments show and describe electrodes 108 for monitoring impedance data, it will be understood by those of skill in the art that the system 100 may include any of a variety of sensors and/or sensing mechanisms for monitoring and/or detecting a change in state within the aspiration lumen 106. Alternate sensing mechanisms may be configured to detect other electrical measurements such as conductivity or resistance, optical properties such as absorption or reflection, or mechanical properties such as shear forces or pressure.

Upon successful clearance of the aspiration lumen 106, negative pressure through the aspiration lumen 106 may be resumed after a pause to allow for a sufficient accumulation of air in the stomach before suctioning resumes. As will be described in further detail below, this pause may be preset, programmable and/or modifiable by a user so that the pause may be tailored to a patient's specific needs. For example, different patients may have different stomach anatomy requiring different interaction with feeds, which may require different suction settings.

According to an exemplary embodiment, the catheter 102 may also include a radiopaque marker 142 extending, for example, circumferentially thereabout at a proximal end 144 of the distal portion 124 of the catheter 102. The radiopaque marker 142 enables a visualization thereof for the purpose of determining a placement of the catheter 102 within the patient body (e.g., within the stomach). For example, the radiopaque marker 142 may facilitate determining of a placement of the distal end 126 and/or a relative position of the openings 120 of the feed lumen 104 and/or the openings 130 of the aspiration lumen 106. It will be understood by those of skill in the art that although the radiopaque marker 142 is shown and described as a circumferential band extending about a periphery of the catheter 102 at the proximal end 144 of the distal portion 124, the radiopaque marker 142 may have any of a variety of configurations and may be located along a variety of positions along, for example, the distal portion 124, so long as the radiopaque marker 142 is configured to aid in a relative positioning of the catheter 102 within the patient body.

In an exemplary embodiment, the system 100 may also include an in-line manometer or pressure sensor 134 that may be used to provide real time aspiration lumen pressures to the controller 110 driving aspiration the second pump 116 activity. The system 100 may also include additional features such as, for example, an in-line drip catch 136 configured to catch small volumes of liquid aspirate along with vacuum relief valves 138 to limit vacuum forces potentially applied to gastric mucosa. As described above, the first pump 114 of the pump device may include, for example, a syringe pump. The second pump 116 may, according to one exemplary embodiment includes a peristatic pump.

In another exemplary embodiment, the second pump 116 may also include a syringe pump. It will be understood by those of skill in the art, however, that the first and second pumps 114, 116 of the pump device may include any of a variety of pump configurations so long as the pump device is capable of independently and simultaneously driving flow through each of the feed lumen 104 and aspiration lumen 106. It will also be understood by those of skill in the art that although the pump device is described as a dual pump including pump capabilities for two distinct pumps (e.g., 114, 116), the first and second pumps 114, 116 may be configured as two separate pumping devices. In an exemplary embodiment, a single pump device configured to perform gastric decompression (i.e., the second pump 116), may be operated independently of any secondary feeding pump system (i.e., the first pump 114) for delivering nutrition.

The controller 110 may include a computer or other processing device capable of receiving pressure and impedance data to control the pump device and provide independent and simultaneous flow to the feed lumen 104 and the aspiration lumen 106. The controller 110 may be connected and/or include a memory configured to include instructions for controlling the pump device, as will be described in further detail below. The memory may include, for example, a non-transitory computer readable storage medium including instructions that are executable via the controller 110. The controller 110 may be configured to execute computer-executable instructions for operations from applications stored in the memory of the controller 110 to provide functionalities to the system 100.

The controller 110 may also include and/or be connected to a user interface 112 which may display data related to the feeding and/or aspiration of the patient. Data may include, for example, average, peak or minimum gastric pressures, along with any other data which would be of clinical interest. The user interface 112 may also be configured to permit user input of preferences and/or selections related to the functioning of the system 100 including selections of preset modes and/or adjustments of, for example, a feeding rate, feeding volume, threshold impedance values, threshold pressure values, aspiration rate, frequency of aspiration, and/or the manual activation or deactivation of pumping functionalities for the feed lumen 104 and/or aspiration lumen 106. In an exemplary embodiment, the user interface 112 may be configured to provide audiovisual alerts to the user upon, reaching certain triggers and/or thresholds. In yet another exemplar embodiment, the user interface 112 may also be configured to provide an alert of abnormal pressure and/or impedance readings, which may be indicative of, for example, a mispositioned catheter, including within the esophagus or lungs.

It will be understood by those of skill in the art that although the system 100 is shown as including a single computer controller 110, the functionalities described with respect to the system 100 may be achievable via a modular component connected thereto or via more than one controller 110. For example, the system 100 may be comprised of a network of computing systems, each of which includes one or more of the functionalities/capabilities described herein.

The system 100 is capable of providing combined gastric pressure monitoring simultaneous with feeding administration and/or pressure measurements independent of feed administration. This offers an opportunity to provide data regarding intrabdominal pressure trends, collected in a manner that is similar to clinical standards currently employed, that may be precursors to critical illness in the neonate, such as necrotizing enterocolitis. This system 100 therefore could provide therapeutic value through removal of excess gastric air, while also providing additional diagnostic value through routine gastric pressure monitoring.

According to an exemplary method for utilizing the system 100, the catheter 102 is inserted into the stomach of a patient substantially similarly to traditional orogastric feeding tubes—e.g., through the oropharynx and esophagus to the stomach. Upon insertion of the catheter 102, feeding may be commenced through the feed lumen 104 thereof. As described above, feed may be administered to the patient according to a desired feeding rate, which may be input to the controller 110. In an exemplary embodiment, feed is passed distally through the feed lumen 104, out of the openings 120, and into the stomach. According to an exemplary embodiment, an impedance and/or a gastric pressure may be continuously measured via, for example, the electrodes 108 and the pressure sensor 132 and/or the pressure sensor 134, respectively.

As shown in FIG. 5, the impedance and pressure measurements are provided to the controller 110 so that the controller 110 may indicate to the second pump 116 when an aspiration should be activated and/or when the aspiration lumen 106 is blocked and needs to be cleared. An aspiration is activated—e.g., the second pump 116 is triggered to provide a negative pressure therethrough—when the measured gastric pressure exceeds a predetermined threshold range or value. As will be understood by those of skill in the art, threshold values may be preset or, in another exemplary embodiment, be set via the user (e.g., physician) of the system 100, as desired. In another exemplary embodiment, the threshold values may be automatically calibrated and adjusted via the controller 110 during longitudinal data collection and historic measurements. A measured pressure beyond the threshold value indicates an accumulation of excess gas within the stomach of the patient. In addition, in an exemplary embodiment, changes in an aspiration rate (volume/time) of the second pump 116 may be triggered based on changes in measured gastric pressure.

Aspiration continues, drawing the excess gas through the openings 130 and into the aspiration lumen 106 to be removed from the patient body, until the measured impedance indicates a change in state within the aspiration lumen 106 or, in some embodiments, a target pressure threshold is reached. In particular, a drop in impedance will indicate a change from gas to liquid within the aspiration lumen 106, which causes the controller 110 to trigger or instruct the second pump 116 to reverse a flow within the aspiration lumen 106 such that a positive pressure is supplied through the aspiration lumen to clear the liquid therefrom, redepositing the liquid into the stomach. Positive pressure may be supplied through the aspiration lumen 106 until the measured impedance indicates that the blockage has been cleared. Aspiration may then be recommenced after a brief pause. This pause may be a set delay window that may be determined based on a gastric pressure reading, may be set via the user, and/or may be manually ended by a user. Based on the particular patient's needs, the pause may be preselected, adjusted, or modified and may include time values such as, for example, 45 seconds, 5 minutes, 10 minutes, etc. Any adjustments, modifications and/or settings may be inputted to the controller 110 via the user interface 112.

As shown, aspiration continues until liquid is detected in the aspiration lumen 106 or a target pressure threshold is reached, initiating a clearance loop. When liquid is detected in the aspiration lumen 106, the gastric pressure will generally be within an accepted threshold range so that positive pressure may be supplied through the aspiration lumen 106 to redeposit the feed within the aspiration lumen 106 into the stomach. When the gastric pressure, however, remains high—e.g., beyond the accepted threshold, a pause may be initiated before the clearance loop is once again activated. In an exemplary embodiment, for example, a safe operative range of gastric pressure may be between −10 and 10 mmHg to safely regulate the magnitude of forces applied to patient tissues. Alerts may be provided to the user via sound and/or display to indicate detection of liquid in the aspiration lumen 106 and/or an inability to clear the aspiration lumen 106, which may indicate a mechanical failure requiring user intervention.

As described above, upon clearing of the blockage, the second pump 116 may resume aspiration after a brief pause, so long as the impedance data continues to indicate that the aspiration lumen 106 is clear of any blockages—e.g., liquid is not detected therein. This pause may be a predetermined window of time and/or may be determined via the measured gastric pressure. The aspiration loop may continue, as necessary, independently of the administration of feed to the patient. The system 100 is configured so that the user may override any predetermined settings and/or intervene during feed administration and/or aspiration, when necessary—e.g., when it is determined that there may be a mechanical failure or when routine line maintenance is required. In an exemplary embodiment, additional pressure thresholds may be set to trigger temporary tolerance of certain amounts or duration of feed loss (i.e., via an aspiration of liquid) under circumstances indicative of, for example, inappropriately high and/or dangerous gastric pressures.

FIG. 6 illustrates a graph of luminal pressures and impedance obtained during a study of the system 100 under physiologic run conditions. In this study physiologic conditions were replicated using a model stomach system exposed to 5 mmHg CPAP pressures. In particular, the graph of FIG. 6 shows an 18-minute sample of data illustrating activation of aspiration, completion of a feed, detection of liquid in the aspiration lumen, and a subsequent reversal of flow through the aspiration lumen (e.g., reintroduction or air).

A first portion of the graph shows luminal pressures within the feed lumen 104 and the aspiration lumen 106, over time, during a feeding of the patient. In this study, aspiration was triggered on a set timing interval. In particular, aspiration was activated after approximately 3.5 mL of feed was deposited into the stomach. Aspiration is shown first, simultaneous with feeding, and then continuing independently as feeding stops after 7 mL of feed is delivered in the model system. The initial elevated pressures within the stomach are indicative of gaseous distension competing for space with the incoming feed. Under normal conditions, these pressures would continue to increase until the feed could not be tolerated—e.g., until the infant spits up the feed. As shown in FIG. 6, however, initiation of the aspiration resulted in a decrease in gastric pressure which allowed simultaneous feeding to continue until the desired volume of feed (e.g., 7 mL) was deposited into the stomach. After completion of the feeding, the system 100 continues to remove excess air, resulting in falling gastric pressure. Although the study shown in FIG. 6 was triggered on a set timing interval, it will be understood by those of skill in the art, that aspiration via the system 100 may also be activated when luminal pressures indicate elevated gastric pressures within the stomach—i.e., when gastric pressure exceeds a predetermined threshold value.

A second portion of the graph, illustrated in a subplot, shows corresponding results of an impedance sensing during the study of the system 100. The sudden drops in impedance during the aspiration indicate liquid (e.g., liquid feed) in the aspiration lumen 106. At this point, a reversal of pressure within the aspiration lumen 106 is initiated to clear the aspiration lumen 106 of the liquid, which is indicated via an increase in the measured impedance. The reversal of pressure (reintroduction of air) enables ongoing cycles of aspiration, followed by line clearance of formula.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

What is claimed is:

1. A device for providing a feeding and aspiration to a patient, comprising:
a catheter extending longitudinally from a proximal end to a distal end;
a feed lumen extending longitudinally through the catheter and including at least one feed opening extending through an exterior wall of the catheter along a distal portion thereof so that the feed lumen is open to an exterior of the catheter via the at least one feed opening;
an aspiration lumen extending longitudinally through the catheter and separated from the feed lumen along a length thereof via a separating wall, the aspiration lumen including a plurality of aspiration openings extending laterally through the exterior wall of the catheter along the distal portion, so that the aspiration lumen is open to the exterior of the catheter via the plurality of aspiration openings; and
a sensor positioned in communication with the aspiration lumen and configured to detect a change in state therewithin.

2. The device of claim 1, wherein the sensor includes a plurality of electrodes configured to measure an impedance within the aspiration lumen.

3. The device of claim 2, wherein each of the plurality of electrodes is configured as a wire electrode embedded in one of the separating wall and the exterior wall so that, a distal portion of which is exposed to an interior of the aspiration lumen, immediately proximal of the distal portion of the catheter.

4. The device of claim 2, further comprising a third lumen extending through the catheter, the plurality of electrodes extending through the third lumen so that distal portions thereof extend through a wall separating the third lumen from the aspiration lumen so that distal portions of the electrodes are exposed to an interior of the aspiration lumen.

5. The device of claim 1, wherein the aspiration lumen extends from the proximal end of the catheter to a distal end that is proximal a distal end of the feed lumen.

6. The device of claim 1, wherein a cross-section of the feed lumen is substantially circular.

7. The device of claim 6, wherein the aspiration lumen extends about a portion of a periphery of the feed lumen.

8. A system for providing a feeding and aspiration to a patient, comprising:
a catheter extending longitudinally from a proximal end to a distal end, the catheter including a feed lumen and an aspiration lumen, the feed lumen extending longitudinally through the catheter and including at least one feed opening extending through an exterior wall of the catheter along a distal portion thereof so that the feed lumen is open to an exterior of the catheter via the at least one feed opening, the aspiration lumen extending longitudinally through the catheter and separated from the feed lumen along a length thereof via a separating wall, the aspiration lumen including a plurality of aspiration openings extending laterally through the exterior wall of the catheter along the distal portion so that the aspiration lumen is open to the exterior of the catheter via the aspiration openings:
a sensing mechanism configured to detect a change in state within the aspiration lumen; and
a controller configured to control a pressure of the aspiration lumen based on a detected change in state within the aspiration lumen.

9. The system of claim 8, wherein the sensing mechanism includes a plurality of electrodes positioned along the aspiration lumen to detect the change in state therein, and an impedance sensor configured to receive input from the plurality of electrodes and to output impedance data indicative of an internal state of the aspiration lumen.

10. The system of claim 8, further comprising:
a first pump configured to provide a flow of liquid feed distally through the feed lumen and out of the at least one feed opening.

11. The system of claim 8, further comprising:
a second pump configured to control the pressure of the aspiration lumen.

12. The system of claim 10, wherein the first pump is a syringe pump.

13. The system of claim 11, wherein the second pump is configured to provide a negative pressure gradient through the aspiration lumen during an aspiration and a positive pressure gradient through the aspiration lumen when the sensing mechanism detects a change in state within the aspiration lumen, from a gas to a liquid.

14. The system of claim 11, wherein the second pump is a peristaltic pump.

15. The system of claim 8, further comprising:
a pressure sensor positioned at a proximal end of the catheter for measuring a gastric pressure.

16. A method for providing independent and simultaneous feeding and aspiration to a patient, comprising:
providing a liquid feed to the patient via a feed lumen extending longitudinally through a catheter positioned within a stomach of the patient, the liquid feed passing distally through the feed lumen and laterally out of the feed lumen via at least one feed opening extending through an exterior wall of the catheter;
activating, via a controller, an aspiration through an aspiration lumen extending longitudinally through the catheter and via a plurality of aspiration openings extending through the exterior wall of the catheter to draw excess gas through the plurality of aspiration openings, proximally out of the aspiration lumen;
continuously determining, via the controller, an impedance data indicating an internal state of the aspiration lumen; and
when the impedance data indicates a change in state within the aspiration lumen from a gas to a liquid, reversing a flow of pressure through the aspiration lumen to clear the aspiration lumen.

17. The method of claim 16, wherein the aspiration is controlled based on whether a measured gastric pressure exceeds a predetermined threshold value.

18. The method of claim 16, wherein the impedance data is determined based on input received from a plurality of electrodes, a portion of which is positioned along an interior of the aspiration lumen.

19. The method of claim 16, wherein a reversal of flow to clear the aspiration lumen ends when the impedance data indicates that liquid is no longer detected in the aspiration lumen.

20. The method of claim 19, further comprising:
reactivating a negative pressure gradient through the aspiration lumen after a pause.

21. The method of claim 20, wherein the pause is determined via one of a predetermined delay window and a measured gastric pressure.

22. The method of claim 16, further comprising:
overriding a reversal of flow in the aspiration lumen for variable periods of time, based on one of user input and a gastric pressure threshold.

23. The method of claim 22, wherein an impedance threshold value is automatically calibrated based on historical impedance measurements.
